(19) United States
(12) Patent Application Publication
(10) Pub. No.: US 2012/0149868 A1
(43) Pub. Date: Jun. 14, 2012

(54) PURIFICATION OF PEPTIDES PREPARED BY SOLID PHASE SYNTHESIS

(75) Inventors: Camilla Kornbeck, Birkeroed (DK); Thomas Budde Hansen, Copenhagen N (DK)

(73) Assignee: Novo Nordisk A/S, Bagsvaerd (DK)

(21) Appl. No.: 12/992,773

(22) PCT Filed: May 15, 2009
(86) PCT No.: PCT/EP2009/055908

§ 371 Date: Feb. 4, 2011

Related U.S. Application Data
(60) Provisional application No. 61/128,258, filed on May 20, 2008.

(30) Foreign Application Priority Data
May 15, 2008 (EP) 08156221.7

(51) Int. Cl. C07K 1/18; C07K 14/605; C07K 2/00; C07K 1/14

(52) U.S. Cl. 530/308; 530/344; 530/300

(57) ABSTRACT

The invention relates to an effective process for purifying a peptide which has been prepared by solid phase peptide synthesis. Also encompassed by the invention is a kit comprising reagents for said process and the purified peptide obtained by said process.

### FIELD OF THE INVENTION

The invention relates to a process for purifying a peptide prepared by solid phase peptide synthesis, to a kit comprising reagents for said process and to the purified peptide obtained by said process.

### BACKGROUND OF THE INVENTION

Polypeptides are increasingly being used as medicaments for the treatment of diseases within all major therapy areas. Polypeptides for therapeutic applications are to be highly purified in order to be efficacious and in order to provide certainty for not causing adverse events upon administration to patients.

One method of obtaining a therapeutic peptide is by solid phase peptide synthesis. The product of solid phase synthesis is a peptide bound to an insoluble support. Peptides synthesized in this manner are then cleaved from the resin, and the cleaved peptide is isolated.

To avoid side reactions during solid phase peptide synthesis, the amine group is masked with an amino terminal protecting group during the coupling reaction (also referred to as an N-terminal protecting group) which includes a chemical moiety coupled to the alpha amino group of an amino acid. Typically, the amino terminal protecting group is removed in a deprotection reaction prior to the addition of the next amino acid to be added to the growing peptide chain, but can be maintained when the peptide is cleaved from the support during solid phase synthesis. The amino terminal group can be maintained when washing or otherwise processing the peptide as well.

Crude peptide mixtures from solid phase peptide synthesis comprise a large number of organic solvents, reagents and process related impurities. Often, the peptide mixture exhibits high UV-absorption which interferes with in-line UV measurements during chromatographic control. Furthermore, a majority of the impurities reduce the capacity of ion exchangers and are difficult to completely remove. Consequently, levels of impurities are typically present even after purification.

9-Fluorenylmethyloxycarbonyl (Fmoc) is an example of a preferred N-terminal protecting group. Fmoc is a base-sensitive N-terminal protecting group which can be de-coupled from the amino acid by a base.

It is known that dibenzofulvene (DBF) is created during cleavage of protecting groups such as Fmoc and subsequent removal is complicated and expensive. There is therefore a great need for an effective purification process following solid phase peptide synthesis.

### SUMMARY OF THE INVENTION

According to a first aspect of the invention there is provided a process for purifying a peptide prepared by solid phase peptide synthesis which comprises the step of bringing a crude extract of the peptide prepared by solid phase peptide synthesis in contact with a solid support.

According to a second aspect of the invention there is provided a solid phase peptide synthesis kit which comprises reagents for solid phase peptide synthesis, a solid support as defined herein and instructions to use said kit in accordance with the process defined herein.

According to a third aspect of the invention there is provided a peptide obtained by a process described herein.

### BRIEF DESCRIPTION OF THE DRAWINGS

FIG. 1. Chromatogram obtained by control purification of a peptide prepared by solid phase synthesis.

FIG. 2. Chromatogram obtained following storage of a peptide prepared by solid phase synthesis in a container comprising polyethylene.

FIG. 3. Chromatogram obtained following chromatographic separation of a peptide prepared by solid phase synthesis in the presence of 100% ethanol.

### DESCRIPTION OF THE INVENTION

The following is a detailed definition of the terms used in the specification.

The term “buffer” as used herein refers to a chemical compound that reduces the tendency of pH of a solution such as chromatographic solutions to change over time as would otherwise occur. Buffers include the following non-limiting examples: sodium acetate, sodium carbonate, sodium citrate, glycylglycine, glycine, histidine, lysine, sodium phosphate, borate, Trishydroxymethyl-aminomethane, ethanolamine and mixtures thereof.

The term “polypeptide” or “peptide” as used interchangeably herein means a compound composed of at least five constituent amino acids connected by peptide bonds. The constituent amino acids may be from the group of the amino acids encoded by the genetic code and they may be natural amino acids which are not encoded by the genetic code, as well as synthetic amino acids. The 22 encoded (also called proteogenic) amino acids are: Alanine, Arginine, Asparagine, Aspartic acid, Cysteine, Cystine, Glutamine, Glutamic acid, Glycine, Histidine, Hydroxyproline, Isoleucine, Leucine, Lysine, Methionine, Phenylalanine, Proline, Serine, Threonine, Tryptophan, Tyrosine, Valine. Natural amino acids which are not encoded by the genetic code but which can be incorporated into a peptide via peptide bonds may be designated as natural non-proteogenic amino acids and are e.g. γ-carboxyglutamate, ornithine, phosphoserine, D-alanine and D-glutamine. Synthetic non-proteogenic amino acids comprise amino acids manufactured by chemical synthesis, i.e. D-isomers of the amino acids encoded by the genetic code such as D-alanine and D-leucine, Aib (α-aminoisobutyric acid), Abu (a-aminobutyric acid), Tle (tert-butylglycine), 3-aminomethyl benzoic acid, anthranilic acid, the beta analogs of amino acids such asp-alanine etc., D-histidine, desamino-histidine, 2-amino-histidine, (β-hydroxy-histidine, homohistidine, N^{α}-acetyl-histidine, α-fluoromethyl-histidine, α-methyl-histidine, 3-pyridylalanine, 2-pyridylalanine or 4-pyridylalanine, (1-aminocyclopropyl)carboxylic acid, (1-aminocyclobutyl)carboxylic acid, (1-aminocyclopentyl)carboxylic acid, (1-aminocyclohexyl)carboxylic acid, (1-aminocycloheptyl)carboxylic acid, or (1-aminocyclooctyl)carboxylic acid.

A polypeptide may comprise a single peptide chain or it may comprise more than one peptide chain, such as e.g. human insulin where two chains are connected by disulphide bonds.

The term “glucagon-like peptide” as used herein refers to the exendins such as exendin-3 and exendin-4 as well as the homologous peptides besides glucagon which are derived from the preproglucagon gene, i.e. glucagon-like peptide 1 (GLP-1), glucagon-like peptide 2 (GLP-2) and oxyntomodulin (OXM) as well as analogues and derivatives thereof. The exendins, which are found in the Gila monster are homologous to GLP-1 and also exert an insulinotropic effect. Examples of exendins are exendin-4 and exendin-3. The glucagon-like peptides have the sequences shown in SEQ ID Nos. 1-6:

### Glucagon (SEQ ID NO: 1);

### GLP-1 (SEQ ID NO: 2);

### GLP-2 (SEQ ID NO: 3);

### Exendin-4 (SEQ ID NO: 4);

### Exendin-3 (SEQ ID NO: 5); and

### OXM (SEQ ID NO: 6).

The term “analogue” as used herein referring to a peptide means a modified peptide wherein one or more amino acid residues of the peptide have been substituted by other amino acid residues and/or wherein one or more amino acid residues have been deleted from the peptide and or wherein one or more amino acid residues have been added to the peptide. Such addition or deletion of amino acid residues can take place at the N-terminal of the peptide and/or at the C-terminal of the peptide. Two different and simple systems are often used to describe analogues: For example Arg³⁴-GLP-1(7-37) or K34R-GLP-1(7-37) designates a GLP-1 analogue wherein the naturally occurring lysine at position 34 has been substituted with arginine (standard single or three letter abbreviation for amino acids used according to IUPAC-IUB nomenclature). All amino acids for which the optical isomer is not stated is to be understood to mean the L-isomer.

In embodiments of the invention a maximum of 17 amino acids have been modified. In embodiments of the invention a maximum of 15 amino acids have been modified. In embodiments of the invention a maximum of 10 amino acids have been modified. In embodiments of the invention a maximum of 8 amino acids have been modified. In embodiments of the invention a maximum of 7 amino acids have been modified. In embodiments of the invention a maximum of 6 amino acids have been modified. In embodiments of the invention a maximum of 5 amino acids have been modified. In embodiments of the invention a maximum of 4 amino acids have been modified. In embodiments of the invention a maximum of 3 amino acids have been modified. In embodiments of the invention a maximum of 2 amino acids have been modified. In embodiments of the invention 1 amino acid has been modified.

The term “derivative” as used herein in relation to a peptide means a chemically modified peptide or an analogue thereof, wherein at least one substituent is not present in the unmodified peptide or an analogue thereof, i.e. a peptide which has been covalently modified. Typical modifications are amides, carbohydrates, alkyl groups, acyl groups, esters, pegylations and the like. An example of a derivative of GLP-1(7-37) is N^{ε26}-((4S)-4-(hexadecanoylamino)-carboxy-butanoyl)[Arg³⁴, Lys²⁶]GLP-1-(7-37).

The term “a fragment thereof” as used herein in relation to a peptide means any fragment of the peptide having at least 20% of the amino acids of the parent peptide. Thus, for human serum albumin a fragment would comprise at least 117 amino acids as human serum albumin has 585 amino acids. In one embodiment the fragment has at least 35% of the amino acids of the parent peptide. In another embodiment the fragment has at least 50% of the amino acids of the parent peptide. In another embodiment the fragment has at least 75% of the amino acids of the parent peptide.

The term “variant” as used herein in relation to a peptide means a modified peptide which is an analog of the parent peptide, a derivative of the parent peptide or a derivative of an analog of the parent peptide.

The term “GLP-1 peptide” as used herein means GLP-1(7-37), an analogue of GLP-1(7-37), a derivative of GLP-1(7-37) or a derivative of a GLP-1(7-37) analogue.

The term “GLP-2 peptide” as used herein means GLP-2(1-33), an analogue of GLP-2, a derivative of GLP-2(1-33) or a derivative of a GLP-2(1-33) analogue.

The term “exendin-4 peptide” as used herein means exendin-4(1-39), an exendin-4 analogue, an exendin-4 derivative or a derivative of an exendin-4 analogue.

The term “plasma stable” glucagon-like peptide such as GLP-1, GLP-2, Glucagon, Exendin-3 or Exendin-4 as used herein means a chemically modified glucagon-like peptide, i.e. an analogue or a derivative of e.g. GLP-1, GLP-2, Glucagon, Exendin-3 or Exendin-4 which exhibits an in vivo plasma elimination half-life of at least 10 hours in man, as determined by the following method. The method for determination of plasma elimination half-life of a glucagon-like peptide in man is: The compound is dissolved in an isotonic buffer, pH 7.4, PBS or any other suitable buffer. The dose is injected peripherally, preferably in the abdominal or upper thigh. Blood samples for determination of active compound are taken at frequent intervals, and for a sufficient duration to cover the terminal elimination part (e.g. Pre-dose, 1, 2, 3, 4, 5, 6, 7, 8, 10, 12, 24 (day 2), 36 (day 2), 48 (day 3), 60 (day 3), 72 (day 4) and 84 (day 4) hours post dose). Determination of the concentration of active compound is performed as described in Wilken et al., Diabetologia 43(51):A143, 2000. Derived pharmacokinetic parameteres are calculated from the concentration-time data for each individual subject by use of non-compartmental methods, using the commercially available software WinNonlin Version 2.1 (Pharsight, Cary, N.C., USA). The terminal elimination rate constant is estimated by log-linear regression on the terminal log-linear part of the concentration-time curve, and used for calculating the elimination half-life.

The term “insulinotropic agent” as used herein means a compound which is an agonist of the human GLP-1 receptor, i.e. a compound which stimulates the formation of cAMP in a suitable medium containing the human GLP-1 receptor (one such medium disclosed below). The potency of an insulinotropic agent is determined by calculating the EC₅₀ value from the dose-response curve as described below.

Baby hamster kidney (BHK) cells expressing the cloned human GLP-1 receptor (BHK-467-12A) were grown in DMEM media with the addition of 100 IU/mL penicillin, 100 μg/mL streptomycin, 5% fetal calf serum and 0.5 mg/mL Geneticin G-418 (Life Technologies). The cells were washed twice in phosphate buffered saline and harvested with Versene. Plasma membranes were prepared from the cells by homogenisation with an Ultraturrax in buffer 1 (20 mM HEPES-Na, 10 mM EDTA, pH 7.4). The homogenate was centrifuged at 48,000×g for 15 min at 4° C. The pellet was suspended by homogenization in buffer 2 (20 mM HEPES-Na, 0.1 mM EDTA, pH 7.4), then centrifuged at 48,000×g for 15 min at 4° C. The washing procedure was repeated one more time. The final pellet was suspended in buffer 2 and used immediately for assays or stored at −80° C.

The functional receptor assay was carried out by measuring cyclic AMP (cAMP) as a response to stimulation by the insulinotropic agent. cAMP formed was quantified by the AlphaScreen™ cAMP Kit (Perkin Elmer Life Sciences). Incubations were carried out in half-area 96-well microtiter plates in a total volume of 50 μL buffer 3 (50 mM Tris-HCl, 5 mM HEPES, 10 mM MgCl₂, pH 7.4) and with the following addiditions: 1 mM ATP, 1 μM GTP, 0.5 mM 3-isobutyl-1-methylxanthine (IBMX), 0.01% Tween-20, 0.1% BSA, 6 μg membrane preparation, 15 μg/mL acceptor beads, 20 μg/mL donor beads preincubated with 6 nM biotinyl-cAMP. Compounds to be tested for agonist activity were dissolved and diluted in buffer 3. GTP was freshly prepared for each experiment. The plate was incubated in the dark with slow agitation for three hours at room temperature followed by counting in the Fusion™ instrument (Perkin Elmer Life Sciences). Concentration-response curves were plotted for the individual compounds and EC₅₀ values estimated using a four-parameter logistic model with Prism v. 4.0 (GraphPad, Carlsbad, Calif.).

The term “DPP-IV protected glucagon-like peptide” as used herein means a glucagon-like peptide which is chemically modified as compared to the natural peptide to render said glucagon-like peptide more resistant to the plasma peptidase dipeptidyl aminopeptidase-4 (DPP-IV).

Resistance of a peptide to degradation by dipeptidyl aminopeptidase IV is determined by the following degradation assay:

Aliquots of the peptide (5 nmol) are incubated at 37° C. with 1 μL of purified dipeptidyl aminopeptidase IV corresponding to an enzymatic activity of 5 mU for 10-180 minutes in 100 μL of 0.1 M triethylamine-HCl buffer, pH 7.4. Enzymatic reactions are terminated by the addition of 5 μL of 10% trifluoroacetic acid, and the peptide degradation products are separated and quantified using HPLC analysis. One method for performing this analysis is:

The mixtures are applied onto a Vydac C18 widepore (30 nm pores, 5 μm particles) 250×4.6 mm column and eluted at a flow rate of 1 ml/min with linear stepwise gradients of acetonitrile in 0.1% trifluoroacetic acid (0% acetonitrile for 3 min, 0-24% acetonitrile for 17 min, 24-48% acetonitrile for 1 min) according to Siegel et al., Regul. Pept. 1999; 79:93-102 and Mentlein et al. Eur. J. Biochem. 1993; 214:829-35. Peptides and their degradation products may be monitored by their absorbance at 220 nm (peptide bonds) or 280 nm (aromatic amino acids), and are quantified by integration of their peak areas related to those of standards. The rate of hydrolysis of a peptide by dipeptidyl aminopeptidase IV is estimated at incubation times which result in less than 10% of the peptide being hydrolysed.

The term “immunomodulated exendin-4 compound” as used herein means an exendin-4 peptide which is an analogue or a derivative of exendin-4(1-39) having a reduced immune response in humans as compared to exendin-4(1-39). The method for assessing the immune response is to measure the concentration of antibodies reactive to the exendin-4 compound after 4 weeks of treatment of the patient.

The term “insulin peptide” as used herein means a peptide which is either human insulin, a human insulin analogue or a chemically modified human insulin, i.e. a derivative of human insulin or a human insulin analogue.

The term “human insulin” as used herein means the human hormone whose structure and properties are well known. Human insulin has two polypeptide chains that are connected by disulphide bridges between cysteine residues, namely the A-chain and the B-chain. The A-chain is a 21 amino acid peptide and the B-chain is a 30 amino acid peptide, the two chains being connected by three disulphide bridges: one between the cysteines in position 6 and 11 of the A-chain, the second between the cysteine in position 7 of the A-chain and the cysteine in Position 7 of the B-chain, and the third between the cysteine in position 20 of the A-chain and the cysteine in position 19 of the B-chain.

The term “polypeptide product” as used herein means the purified peptide product which is to be used for the manufacture of a pharmaceutical composition. Thus, the polypeptide product is normally obtained as the product from the final purification, drying or conditioning step. The product may be crystals, precipitate, solution or suspension. The polypeptide product is also known in the art as the drug substance, i.e. the active pharmaceutical ingredient.

The term “isoelectric point” as used herein means the pH value where the overall net charge of a macromolecule such as a polypeptide is zero. In polypeptides there may be many charged groups, and at the isoelectric point the sum of all these charges is zero. At a pH above the isoelectric point the overall net charge of the polypeptide will be negative, whereas at pH values below the isoelectric point the overall net charge of the polypeptide will be positive.

The term “pharmaceutically acceptable” as used herein means suited for normal pharmaceutical applications, i.e. giving rise to no adverse events in patients.

The term “excipient” as used herein means the chemical compounds which are normally added to pharmaceutical compositions, e.g. buffers, tonicity agents, preservatives and the like.

The term “effective amount” as used herein means a dosage which is sufficient to be effective for the treatment of the patient compared with no treatment.

The term “pharmaceutical composition” as used herein means a product comprising an active compound or a salt thereof together with pharmaceutical excipients such as buffer, preservative, and optionally a tonicity modifier and/or a stabilizer. Thus a pharmaceutical composition is also known in the art as a pharmaceutical formulation.

The term “treatment of a disease” as used herein means the management and care of a patient having developed the disease, condition or disorder. The purpose of treatment is to combat the disease, condition or disorder. Treatment includes the administration of the active compounds to eliminate or control the disease, condition or disorder as well as to alleviate the symptoms or complications associated with the disease, condition or disorder.

It will be appreciated that solid phase peptide synthesis is well known to the person skilled in the art. Furthermore, coupling and subsequent deprotection steps of a protecting group from a peptide are also well known to the person skilled in the art.

In one embodiment, solid phase peptide synthesis comprises the use of Fmoc as an amino-terminal protecting group. In a further embodiment, the purification process of the invention comprises a process for removing dibenzofulvene from the crude peptide extract.

In one embodiment, the deprotection step of the solid phase peptide synthesis product comprises the use of a base. In a further embodiment, the base is selected from a secondary amine and/or a reagent capable of hydrogenolysis. In a yet further embodiment, the base is selected from piperidine, diethylamine and piperazine.

In one embodiment, the deprotection step of the solid phase peptide synthesis product is performed in a solvent, such as N-methylpyrrolidone (NMP), dimethylformamide (DMF) and dichloromethane (DCM).

In one embodiment, the purification of the crude peptide extract obtained after the deprotection step comprises applying the crude peptide extract to a solid support followed by eluting the purified product there from.

In one embodiment, the solid support comprises an ion-exchange chromatographic column. In a further embodiment, the solid support comprises an anionic resin or a cationic resin. In a still further embodiment, the solid support comprises a resin selected from the group consisting of Source 30Q, Poros 50HQ, Q Sepharose HP, Q Ceramic HyperD F. In a yet further embodiment the solid support comprises an anionic resin (e.g. a quaternary ammonium resin such as Source 30Q).

In the embodiment of the invention wherein the solid support comprises an ion-exchange chromatographic column, the purification process of the invention comprises the following steps:

(a) loading the ion-exchange chromatographic column with the crude peptide extract obtained from solid-phase synthesis under standard chromatographic conditions;
(b) performing an elution step with an alcohol; and
(c) performing chromatographic separation with one or more buffers.

In one embodiment the alcohol in step (b) is a C₁₋₅ alcohol, i.e. an alcohol having from between 1 to 5 carbon atoms. In another embodiment the alcohol is a C₁₋₃ alcohol. In one embodiment the alcohol is an unbranched or branched alcohol selected from the group consisting of: methanol, ethanol, 1-propanol (propanol), 2-propanol (isopropyl alcohol), 2-methyl-1-propanol (isobutyl alcohol), 2-methyl-2-propanol (tert-butyl alcohol), 1-butanol (butanol), 2-butanol, 2-methyl-1-butanol, 3-methyl-1-butanol, 2-methyl-2-butanol, 3-methyl-2-butanol, 1-pentanol (pentanol), 2-pentanol and 3-pentanol. In a further embodiment the alcohol is selected from the group consisting of ethanol and propanol. In one embodiment the alcohol in step (b) is selected from the group consisting of: 70% ethanol, 80% ethanol, 90% ethanol (in water) and 100% ethanol. In one embodiment the alcohol in step (b) is 100% ethanol.

This embodiment of the invention provides the advantage that the elution step (step (b)) efficiently removes impurities from the ion-exchange column. For example, when Fmoc has been used as an N-terminal protecting group during peptide synthesis, it has been surprisingly found that this step selectively elutes dibenzofulvene. The chromatographic separation step (step (c)) subsequently separates the purified peptide in the absence of any residual impurity (e.g. dibenzofulvene) as demonstrated herein.

Examples of buffers which may be used in step (c) include Tris (tris(hydroxymethyl)methylamine), TAPS (3-{[tris(hydroxymethyl)methyl]amino}propanesulfonic acid), Bicine (N,N-bis(2-hydroxyethyl)glycine), Tricine (N-tris(hydroxymethyl)methylglycine), HEPES (4-2-hydroxyethyl-1-piperazineethanesulfonic acid), TES (2-{[tris(hydroxymethyl)methyl]amino}ethanesulfonic acid), MOPS (3-(N-morpholino)propanesulfonic acid), PIPES (piperazine-N,N′-bis(2-ethanesulfonic acid)), Cacodylate (dimethylarsinic acid), MES (2-(N-morpholino)ethanesulfonic acid) or acetate. In one embodiment, the buffer used in step (c) comprises Tris buffer (e.g. 0.02 mol/kg Tris buffered to pH 8.0).

It will be appreciated that the buffers may be used in step (c) optionally in the presence of one or more solvents (e.g. ethanol, such as 50% (w/w) ethanol). It will also be appreciated that the conditions for separation step (c) will typically be those known to a person skilled in the art. For example, equilibration with a first buffer followed by elution by application of a linear gradient from the first buffer to a second buffer (which will typically be the same as the first buffer apart from the presence of one or more salts (e.g. sodium chloride, such as 0.0625 mol/kg sodium chloride)).

In one embodiment, the solid support comprises a packaging material such as a container, pellets, particles or a filter-support comprising a thermoplastic polymer such as polyethylene, polypropylene, polystyrene or a similar material. In another embodiment, the solid support comprises a packaging material such as a container, pellets, particles or a filter-support comprising polyethylene, polypropylene or polystyrene. In yet another embodiment, the solid support comprises a packaging material such as a container, pellets, particles or a filter-support comprising polyethylene.

When used herein the term “container” shall mean any means used to contain the peptide to be purified, before or after purification.

In the embodiment of the invention wherein the solid support comprises a packaging material comprising a thermoplastic polymer, the purification process of the invention comprises the following steps:

(a) addition of the crude peptide extract obtained from solid-phase synthesis to the packaging material;
(b) incubation of the extract in the packaging material;
(c) removal of the extract from the packaging material; and
(d) subjecting the extract to standard peptide separation.

When used herein the term “standard peptide separation” shall mean any separation method known in the art suitable for separating peptides from impurities such as chromatographic separation (such as ion exchange chromatography, hydrophobic interaction chromatography or reversed phase HPLC (High Performance Liquid Chromatography)), Ultra Filtration (UF), iso-electric precipitation or any other suitable separation method.

In one embodiment, standard peptide separation in step (d) is ion-exchange chromatography such as anion-exchange chromatography.

References to “polyethylene”, “polypropylene” etc. include references to a polymer consisting of a plurality (i.e. more than one) monomer units of ethylene (IUPAC name ethene), propylene (IUPAC name propene), etc. For exemplification the general structure of polyethylene is shown below as a compound of formula (I), where n is an integer:

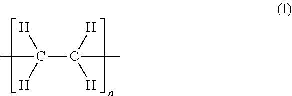

The polyethylene may be in the form of high density polyethylene (HDPE) or low density polyethylene (LDPE). In one embodiment, the polyethylene is high density polyethylene (HDPE). HDPE has a low degree of branching and thus stronger intermolecular forces and tensile strength and is defined by a density of greater or equal to 0.941 g/cm3. LDPE is defined by a density range of 0.910-0.940 g/cm3.

The polypropylene may be in the form of high density polypropylene (HDPP) or low density polypropylene (LDPP). In one embodiment, the polypropylene is high density polypropylene (HDPP).

This embodiment of the invention provides the advantage that addition of the crude peptide extract to the defined packaging material results in adherence of impurities to the surface of the packaging material. Thus, the process efficiently removes impurities from the crude peptide extract. For example, when Fmoc has been used as an N-terminal protecting group during peptide synthesis, it has been surprisingly found that this step selectively adheres dibenzofulvene to the surface of the polyethylene packaging material as demonstrated herein.

In one embodiment, the incubation step (b) typically comprises incubation at an ambient temperature (e.g. room temperature) for a duration of between 2 minutes and 10 hours. In another embodiment, the duration is between 2 minutes and 2 hours. In yet another embodiment the duration is between 2 minutes and 30 minutes.

In one embodiment, step (b) may additionally comprise agitation of the extract.

In one embodiment, step (d) typically comprises chromatographic separation in accordance with known procedures which may include batch absorption, a packed column or a filter.

In one embodiment step (d) comprises chromatographic separation wherein a packed column or a filter is used, and wherein the residence time is at least 0.1 minutes. In another embodiment the residence time is at least 1 minute. In yet another embodiment the residence time is between 0.1 minutes and 60 minutes. In still another embodiment the residence time is between 1 minute and 10 minutes.

When used herein the term “residence time” is to be understood as the average time the peptide is in contact with the packaging material, i.e. how fast the peptide moves through the packaging material.

In one embodiment of the present invention the polypeptide is a glucagon-like peptide.

In one embodiment of the present invention the glucagon-like peptide is a DPP-IV protected glucagon-like peptide.

In one embodiment of the present invention the glucagon-like peptide is a plasma stable glucagon-like peptide.

In one embodiment of the present invention the glucagon-like peptide has a lysine residue, such as one lysine, wherein a lipophilic substituent optionally via a spacer is attached to the epsilon amino group of said lysine.

In one embodiment, the lipophilic substituent comprises an acyl group. An acyl group has the formula, R(C═O)—.

In one embodiment of the present invention the lipophilic substituent has from 8 to 40 carbon atoms, preferably from 8 to 24 carbon atoms, e.g. 12 to 18 carbon atoms.

In embodiments the invention provides a derivative of a glucagon-like peptide comprising a lipophilic substituent, wherein the lipophilic substitutent comprises a straight-chain or branched alkane α,ω-dicarboxylic acid.

In one embodiment the invention provides a glucagon-like peptide according to the embodiments above, wherein the lipophilic substitutent is or comprises a moiety selected from the group consisting of CH₃—(CH₂)ₙ—CO—, (COOH)—(CH₂)ₙ—CO—, (COOH)—(CH₂)ₙ—CO—NH—(CH₂)ₘ—R—CO—, (NH₂—CO)—(CH₂)ₙ—CO— and HO—(CH₂)ₙ—CO—; wherein R is a cycloalkyl selected from the group consisting of cyclopentyl, cyclohexyl and cycloheptyl, 4≦n≦38 and 0≦m≦4.

In one embodiment 12≦n≦36. In one embodiment 12≦n≦20.

In one embodiment, the lipophilic substituent is selected from the group consisting of CH₃—(CH₂)ₙ—CO—, (COOH)—(CH₂)ₙ—CO—, (COOH)—(CH₂)ₙ—CO—NH—(CH₂)ₘ—R—CO—, (NH₂—CO)—(CH₂)ₙ—CO—, HO—(CH₂)ₙ—CO—; wherein R is a cycloalkyl selected from the group consisting of cyclopentyl, cyclohexyl and cycloheptyl, 4≦n≦38 and 1≦m≦4.

In one embodiment, the lipophilic substituent is selected from the group consisting of CH₃—(CH₂)ₙ—CO—, (COOH)—(CH₂)ₙ—CO—, (COOH)—(CH₂)ₙ—CO—NH—(CH₂)ₘ—R—CO—, (NH₂—CO)—(CH₂)ₙ—CO—, HO—(CH₂)ₙ—CO—; wherein R is cyclohexyl, 12≦n≦20 and 1≦m≦2.

One or more lipophilic substituent may be connected to the active component either directly or via a suitable spacer. In one embodiment, the lipophilic component is attached via a suitable spacer.

In one embodiment, the spacer is present and comprises at least one amino acid residue.

In one embodiment of the present invention the spacer is present and is selected from an amino acid, e.g. beta-Ala, L-Glu or aminobutyroyl.

In one embodiment, the spacer is present and is selected from the group consisting of a γ- or an α-glutamyl linker, a β- or an α-aspartyl linker, an α-amido-γ-glutamyl linker, or an α-amido-β-aspartyl linker, or combinations thereof.

In one embodiment, the spacer is of the general formula I

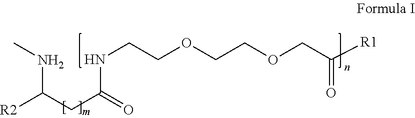

wherein
n is 0-4;
m is 1-2;
R1 designates the attachment site to the active component;

### R2 is COR3 or H; and

R3 is OH, NH₂ or C₁₋₁₂ alkyl, and benzyl.

In one embodiment, the spacer is of the general formula II

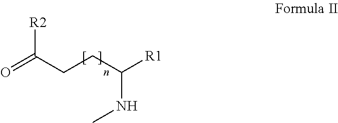

wherein
n is 0-8;

### R1 is COOR3;

R2 designates the attachment site to the active component; and
R3 is selected from hydrogen, C₁₋₁₂-alkyl and benzyl.

In one embodiment of the present invention the polypeptide is glucagon, a glucagon analogue, a derivative of glucagon or a derivative of a glucagon analogue.

In one embodiment of the present invention the glucagon-like peptide is GLP-1, a GLP-1 analogue, a derivative of GLP-1 or a derivative of a GLP-1 analogue.

In one embodiment of the present invention the glucagon-like peptide is a GLP-1 peptide which has from 22 to 40 amino acid residues, preferable from 26 to 36 amino acid residues, even more preferable from 29 to 33 amino acid residues.

In one embodiment of the present invention the GLP-1 peptide is a GLP-1 analogue.

In one embodiment of the present invention the GLP-1 analogue is selected from the group consisting of Arg³⁴-GLP-1(7-37). Gly⁸-GLP-1(7-36)-amide, Gly⁸-GLP-1(7-37), Val⁸-GLP-1(7-36)-amide, Val⁸-GLP-1 (7-37). Val⁸Asp²²-GLP-1(7-36)-amide, Val⁸Asp²²-GLP-1(7-37), Val⁸Glu²²-GLP-1(7-36)-amide, Val⁸Glu²²-GLP-1(7-37), Val⁸Lys²²-GLP-1(7-36)-amide, Val⁸Lys²²-GLP-1(7-37), Val⁸Arg²²-GLP-1(7-36)-amide, Val⁸Arg²²-GLP-1(7-37), Val⁸His²²-GLP-1(7-36)-amide, Val⁸His²²-GLP-1(7-37), Val⁸Trp¹⁹Glu²²-GLP-1(7-37), Val⁸Glu²²Val²⁵-GLP-1(7-37), Val⁸Tyr¹⁶Glu²²-GLP-1(7-37), Val⁸Trp¹⁶Glu²²-GLP-1(7-37), Val⁸Leu¹⁶Glu²²-GLP-1(7-37), Val⁸Tyr¹⁸Glu²²-GLP-1(7-37), Val⁸Glu²²His³⁷-GLP-1(7-37), Val⁸Glu²²Ile³³-GLP-1(7-37), Val⁸Trp¹⁶Glu²²Val²⁵Ile³³-GLP-1(7-37), Val⁸Trp¹⁶Glu²²Ile³³-GLP-1(7-37), Val⁸Glu²²Val²⁵Ile³³-GLP-1(7-37), Val⁸Trp¹⁶Glu²²Val²⁵-GLP-1(7-37), Aib⁸Arg³⁴-GLP-1(7-37), Aib^{8,22}Arg³⁴-GLP-1(7-37), [3-(4-imidazolyl)propionyl]⁷Arg³⁴GLP-1-(7-37), Gly⁸Arg³⁴-GLP-1(7-37), Aib⁸Arg³⁴Pro³⁷-GLP-1(7-37), Aib^{8,22,27,30,35}Arg³⁴Pro³⁷-GLP-1 (7-37)amide, DesaminoHis⁷Glu²²Arg²⁶Arg³⁴Lys³⁷-GLP-1(7-37), Aib⁸Glu²²Arg²⁶Arg³⁴Lys³⁷-GLP-1-(7-37)amide, DesaminoHis⁷Glu²²Arg²⁶Arg³⁴Phe(m-CF3)²⁸-GLP-1-(7-37)amide, Aib⁸Glu²²Arg²⁶Lys³⁰-GLP-1-(7-37), Aib⁸Glu²²Arg²⁶Lys³¹-GLP-1-(7-37), Aib⁸Glu²²Arg²⁶Lys³¹Arg³⁴-GLP-1-(7-37), DesaminoHis⁷Glu²²Arg²⁶Glu³⁰Arg³⁴Lys³⁷-GLP-1-(7-37), Aib⁸Lys²⁰Glu²²Arg²⁶Glu³⁰Pro³⁷-GLP-1-(7-37)amide, Aib⁸Glu²²Arg²⁶Glu³⁰Pro³⁷-GLP-1-(7-37), desaminoHis⁷Glu²²Arg²⁶Glu³⁰Arg³⁴Lys³⁷-GLP-1-(7-37)amide, desaminoHis⁷Glu²²Arg²⁶Arg³⁴Lys³⁷-GLP-1-(7-37)amide, Aib⁸Glu²²Arg²⁶Glu³⁰Lys³⁶-GLP-1-(7-37)Glu-amide, Aib⁸Glu²²Arg²⁶Lys³¹GLP-1-(7-37), analogues thereof and derivatives of any of these.

In one embodiment of the present invention the glucagon-like peptide is a derivative of GLP-1 or a derivative of a GLP-1 analogue which has a lysine residue, such as one lysine, wherein a lipophilic substituent optionally via a spacer is attached to the epsilon amino group of said lysine.

In one embodiment the GLP-1 analogue or derivative is modified in at least one of the amino acid residues in positions 7 and 8 of a GLP-1(7-37) peptide or an analog thereof, and has a lipophilic substituent optionally via a spacer attached to the epsilon amino group on the lysine residue in position 26 of said GLP-1 analogue.

In one embodiment of the present invention the lipophilic substituent has from 8 to 40 carbon atoms, preferably from 8 to 24 carbon atoms, e.g. 12 to 18 carbon atoms.

In embodiments the invention provides a derivative of a GLP-1 peptide comprising a lipophilic substituent, wherein the lipophilic substitutent comprises a straight-chain or branched alkane α,ω-dicarboxylic acid.

In one embodiment the invention provides a GLP-1 peptide according to the embodiments above comprising a lipophilic substituent, wherein the lipophilic substitutent is or comprises a moiety selected from the group consisting of CH₃—(CH₂)ₙ,—CO—, (COOH)—(CH₂)ₙ—CO—, (COOH)—(CH₂), —CO—NH—(CH₂), —R—CO—, (NH₂—CO)—(CH₂)ₙ—CO— and HO—(CH₂)ₙ—CO—; wherein R is a cycloalkyl selected from the group consisting of cyclopentyl, cyclohexyl and cycloheptyl, 4≦n≦38 and 0≦m≦4.

In one embodiment 12≦n≦36. In one embodiment 12≦n≦20.

In one embodiment, the lipophilic substituent is selected from the group consisting of CH₃—(CH₂)ₙ—CO—, (COOH)—(CH₂)ₙ—CO—, (COOH)—(CH₂)ₙ—CO—NH—(CH₂)ₘ—R—CO—, (NH₂—CO)—(CH₂)ₙ—CO—, HO—(CH₂)ₙ—CO—; wherein R is a cycloalkyl selected from the group consisting of cyclopentyl, cyclohexyl and cycloheptyl, 4≦n≦38 and 1≦m≦4.

In one embodiment, the lipophilic substituent is selected from the group consisting of CH₃—(CH₂)ₙ—CO—, (COOH)—(CH₂)ₙ—CO—, (COOH)—(CH₂)ₙ—CO—NH—(CH₂)ₘ—R—CO—, (NH₂—CO)—(CH₂)ₙ—CO—, HO—(CH₂)ₙ—CO—; wherein R is cyclohexyl, 12≦n≦20 and 1≦m≦2.

One or more lipophilic substituent may be connected to the active component either directly or via a suitable spacer. In one embodiment, the lipophilic component is attached via a suitable spacer.

In one embodiment, the spacer is present and comprises at least one amino acid residue.

In one embodiment of the present invention the spacer is present and is selected from an amino acid, e.g. beta-Ala, L-Glu or aminobutyroyl.

In one embodiment, the spacer is present and is selected from the group consisting of a γ- or an α-glutamyl linker, a β- or an α-aspartyl linker, an α-amido-γ-glutamyl linker, or an α-amido-β-aspartyl linker, or combinations thereof.

In one embodiment, the spacer is of the general formula I

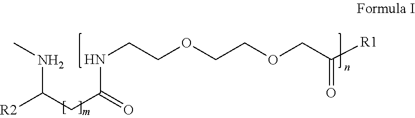

wherein
n is 0-4;
m is 1-2;
R1 designates the attachment site to the active component;

### R2 is COR3 or H; and

R3 is OH, NH₂ or C₁₋₁₂ alkyl, and benzyl.

In one embodiment, the spacer is of the general formula II

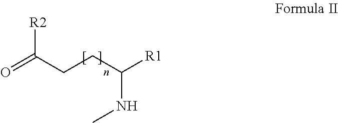

wherein
n is 0-8;

### R1 is COOR3;

R2 designates the attachment site to the active component; and
R3 is selected from hydrogen, C₁₋₁₂-alkyl and benzyl.

In one embodiment of the present invention the GLP-1 peptide is a DPP-IV protected GLP-1 peptide.

In one embodiment of the present invention the GLP-1 peptide is a plasma stable GLP-1 peptide.

In one embodiment of the present invention the glucagon-like peptide is a derivative of a GLP-1 analogue which is selected from the group consisting of: Arg³⁴Lys²⁶(N^{ε}-(γ-Glu(N^{α}-hexadecanoyl)))-GLP-1(7-37), N-ε²⁶-(17-carboxyheptadecanoyl)-[Aib⁸,Arg³⁴]-1-(7-37)-peptide, N-ε²⁶-(19-carboxynonadecanoyl)-[Aib⁸,Arg³⁴]-1-(7-37)-peptide, N-ε²⁶-(4-{[N-(2-carboxyethyl)-N-(15-carboxypentadecanoyl)amino]methyl}benzoyl)[Arg³⁴]-1-(7-37), N-ε²⁶-[2-(2-[2-(2-[2-(2-[4-(17-Carboxyheptadecanoylamino)-4(S)-carboxybutyrylamino]ethoxy)ethoxy]acetylamino)ethoxy]ethoxy)acetyl][Aib⁸,Arg³⁴]GLP-1-(7-37)peptide, N-ε³⁷-{2-[2-(2-{2-[2-((R)-3-carboxy-3-{[1-(19-carboxynonadecanoyl)piperidine-4-carbonyl]amino}propionylamino)ethoxy]ethoxy}acetylamino)ethoxy]ethoxy}acetyl[desaminoHis⁷,Glu²²,Arg²⁶,Arg³⁴,Lys³⁷]GLP-1(7-37)amide; N-ε²⁰-{2-[2-(2-{2-[2-((R)-3-carboxy-3-{[1-(19-carboxynonadecanoyl)piperidine-4-carbonyl]amino}propionylamino)ethoxy]ethoxy}acetylamino)ethoxy]ethoxy}acetyl[Aib²,Leu¹⁴,Lys²⁰,Gln²⁸,Ser(O-Benzyl)³⁹]exendin-4 (1-39)amide; N-ε^{26{}2-[2-(2-{2-[2-((R)-3-carboxy-3-{[1-(19-carboxynonadecanoyl)piperidine-4-carbonyl]amino}propionylamino)ethoxy]ethoxy}acetylamino)ethoxy]ethoxy}acetyl[desaminoHis⁷,Arg³⁴]GLP-1-(7-37); N-ε²⁶-[(S)-4-Carboxy-4-({trans-4-[(19-carboxynonadecanoylamino)methyl]cyclohexanecarbonyl}amino)butyryl][Aib⁸,Arg³⁴]GLP-1-(7-37); N-ε²⁶-{4-[(S)-4-Carboxy-4-({trans-4-[(19-carboxynonadecanoylamino)methyl]cyclohexanecarbonyl}amino)butyrylamino]butyryl}[Aib⁸,Arg³⁴]-1-(7-37); N-ε37-[2-(2-{2-[2-(2-{2-[(S)-4-Carboxy-4-({trans-4-[(19-carboxynonadecanoylamino)methyl]cyclohexanecarbonyl}amino)butyrylamino]ethoxy}ethoxy)acetylamino]ethoxy}ethoxy)acetyl][DesaminoHis⁷,Glu²²,Arg²⁶,Arg³⁴,Lys³⁷]GLP-1-(7-37) and [Aib⁸,Glu²²,Arg²⁶,Glu³⁰,Pro³⁷]GLP-1-((7-37)Lys (2-(2-(3-(2-(2-(2-(2-(2-(2-(2-(2-(2-(2-(2-(2-[4-(S)-carboxy-4-(4-(S)-carboxy-4-(4-{4-[16-(Tetrazol-5-yl)hexadecanoylsulfamoyl]butanoylamino}butanoylamino)butyrylamino)butyrylamino]ethoxy)ethoxy)ethoxy)ethoxy)ethoxy)ethoxy)ethoxy)ethoxy)ethoxy)ethoxy)ethoxy)ethoxy)propionylamino)ethoxy)ethoxy)peptide.

In one embodiment of the present invention the glucagon-like peptide is GLP-2, a GLP-2 analogue, a derivative of GLP-2 or a derivative of a GLP-2 analogue.

In one embodiment of the present invention the derivative of GLP-2 or a derivative of a GLP-2 analogue has a lysine residue, such as one lysine, wherein a lipophilic substituent optionally via a spacer is attached to the epsilon amino group of said lysine.

In one embodiment of the present invention the lipophilic substituent has from 8 to 40 carbon atoms, preferably from 8 to 24 carbon atoms, e.g. 12 to 18 carbon atoms.

In one embodiment of the present invention the spacer is present and is selected from an amino acid, e.g. beta-Ala, L-Glu, or aminobutyroyl.

In one embodiment of the present invention the GLP-2 peptide has from 27 to 39 amino acid residues, preferable from 29 to 37 amino acid residues, even more preferable from 31 to 35 amino acid residues.

In one embodiment of the invention the glucagon-like peptide is Lys¹⁷Arg³⁰-GLP-2(1-33) or Arg³⁰Lys¹⁷(N^{ε}-(β-Ala(N^{α}-hexadecanoyl)))-GLP-2(1-33).

In one embodiment of the present invention the glucagon-like peptide is Gly²-GLP-2(1-33).

In one embodiment of the present invention the glucagon-like peptide is exendin-4, an exendin-4 analogue, a derivative of exendin-4, or a derivative of an exendin-4 analogue.

In one embodiment of the present invention the glucagon-like peptide is exendin-4.

In one embodiment of the present invention the derivative of exendin-4 or derivative of an exendin-4 analogue is an acylated peptide or a pegylated peptide.

In one embodiment of the present invention the glucagon-like peptide is a stable exendin-4 compound.

In one embodiment of the present invention the glucagon-like peptide is a DPP-IV protected exendin-4 compound.

In one embodiment of the present invention the glucagon-like peptide is an immune modulated exendin-4 compound.

In one embodiment of the present invention the derivative of exendin-4 or derivative of an exendin-4 analogue has a lysine residue, such as one lysine, wherein a lipophilic substituent optionally via a spacer is attached to the epsilon amino group of said lysine.

In one embodiment of the present invention the lipophilic substituent has from 8 to 40 carbon atoms, preferably from 8 to 24 carbon atoms, e.g. 12 to 18 carbon atoms.

In one embodiment of the present invention the spacer is present and is selected from an amino acid, e.g. beta-Ala, L-Glu, or aminobutyroyl.

In one embodiment of the present invention the glucagon-like peptide is an exendin-4 peptide which has from 30 to 48 amino acid residues, from 33 to 45 amino acid residues, preferable from 35 to 43 amino acid residues, even more preferable from 37 to 41 amino acid residues.

In one embodiment of the invention the GLP-2 peptide is selected from the list consisting of:

K30R-GLP-2(1-33); S5K-GLP-2(1-33); S7K-GLP-2(1-33); D8K-GLP-2(1-33); E9K-GLP-2(1-33); M10K-GLP-2(1-33); N11K-GLP-2(1-33); T12K-GLP-2(1-33); 113K-GLP-2(1-33); L14KGLP-2(1-33); D15K-GLP-2(1-33); N16K-GLP-2(1-33); L17K-GLP-2(1-33); A18K-GLP-2(1-33); D21K-GLP-2(1-33); N24K-GLP-2(1-33); Q28K-GLP-2(1-33); S5K/K30R-GLP-2(1-33); S7K/K30R-GLP-2(1-33); D8K/K30R-GLP-2(1-33); E9K/K30R-GLP-2(1-33); M10K/K30R-GLP-2(1-33); N11K/K30R-GLP-2(1-33); T12K/K30R-GLP-2(1-33); 113K/K30R-GLP-2(1-33); L14K/K30R-GLP-2(1-33); D15K/K30R-GLP-2(1-33); N16K/K30R-GLP-2(1-33); L17K/K30RGLP-2(1-33); A18K/K30R-GLP-2(1-33); D21K/K30R-GLP-2(1-33); N24K/K30R-GLP-2(1-33); Q28K/K30R-GLP-2(1-33); K30R/D33K-GLP-2(1-33); D3E/K30R/D33E-GLP-2(1-33); D3E/S5K/K30R/D33E-GLP-2(1-33); D3E/S7K/K30R/D33E-GLP-2(1-33); D3E/D8 K/K30R/D33E-GLP-2(1-33); D3E/E9K/K30R/D33E-GLP-2(1-33); D3E/M10K/K30R/D33E-GLP-2(1-33); D3E/N11K/K30R/D33E-GLP-2(1-33); D3E/T12K/K30R/D33E-GLP-2(1-33); D3E/113K/K30R/D33E-GLP-2(1-33); D3E/L14K/K30R/D33E-GLP-2(1-33); D3E/D15K/K30R/D33E-GLP-2(1-33); D3E/N16K/K30R/D33E-GLP-2(1-33); D3E/L17K/K30R/D33E-GLP-2(1-33); D3E/A18K/K30R/D33E-GLP-2(1-33); D3E/D21K/K30R/D33E-GLP-2(1-33); D3E/N24K/K30R/D33E-GLP-2(1-33); D3E/Q28K/K30R/D33E-GLP-2(1-33); and derivatives thereof.

In one embodiment of the invention the GLP-2 derivative is selected from the group consisting of
S5K(3-(hexadecanoylamino)propionyl)-GLP-2(1-33);
S7K(3-(hexadecanoylamino)propionyl)-GLP-2(1-33);
D8K(3-(hexadecanoylamino)propionyl)-GLP-2(1-33);
E9K(3-(hexadecanoylamino)propionyl)-GLP-2(1-33);
M10K(3-(hexadecanoylamino)propionyl)-GLP-2(1-33);
N11K (3-(hexadecanoylamino)propionyl)-GLP-2(1-33);
T12K(3-(hexadecanoylamino)propionyl)-GLP-2(1-33);
I13K(3-(hexadecanoylamino)propionyl)-GLP-2(1-33);
L14K(3-(hexadecanoylamino)propionyl)-GLP-2(1-33);
D15K(3-(hexadecanoylamino)propionyl)-GLP-2(1-33);
N16K(3-(hexadecanoylamino)propionyl)-GLP-2(1-33);
L17K(3-(octanoylamino)propionyl)-GLP-2(1-33);
L17K(3-(nonanoylamino)propionyl)-GLP-2(1-33);
L17K(3-(decanoylamino)propionyl)-GLP-2(1-33);
L17K(3-(undecanoylamino)propionyl)-GLP-2(1-33);
L17K(3-(dodecanoylamino)propionyl)-GLP-2(1-33);
L17K(3-(tridecanoylamino)propionyl)-GLP-2(1-33);
L17K(3-(tetradecanoylamino)propionyl)-GLP-2(1-33);
L17K(3-(pentadecanoylamino)propionyl)-GLP-2(1-33);
L17K(3-(hexadecanoylamino)propionyl)-GLP-2(1-33);
L17K(3-(heptadecanoylamino)propionyl)-GLP-2(1-33);
L17K(3-(octadecanoylamino)propionyl)-GLP-2(1-33);
L17K(3-(nonadecanoylamino)propionyl)-GLP-2(1-33);
L17K(3-(eicosanoylamino)propionyl)-GLP-2(1-33);
L17K((S)-4-carboxy-4-(octanoylamino)butanoyl)-GLP-2(1-33);
L17K((S)-4-carboxy-4-(nonanoylamino)butanoyl)-GLP-2(1-33);
L17K((S)-4-carboxy-4-(decanoylamino)butanoyl)-GLP-2(1-33);
L17K((S)4-carboxy-4-(undecanoylamino)butanoyl)-GLP-2(1-33);
L17K((S)-4-carboxy4-(dodecanoylamino)butanoyl)-GLP-21(1-33);
L17K((S)-4-carboxy-4-(tridecanoylamino)butanoyl)-GLP-2(1-33);
L17K((S˜-carboxy4-(tetradecanoylamino)butanoyl)-GLP-2(1-33);
L17K((S)-4-carboxy-4-(pentadecanoylamino)butanoyl)-GLP-2(1-33);
L17K((S)-4-carboxy-4-(hexadecanoylamino)butanoyl)-GLP-2(1-33);
LI7K((S)-4-carboxy-4-(heptadecanoylamino)butanoyl)-GLP-2(1-33);
L17K((S)-4-carboxy-4-(octadecanoylamino)butanoyl)-GLP-2(1-33);
L17K((S)-4-carboxy-4-(nonadecanoylamino)butanoyl)-GLP-2(1-33);
L17K((S)-4-carboxy-4-(eicosanoylamino)butanoyl)-GLP-2(1-33);
L17K(4-(octanoylamino)butanoyl)-GLP-2(1-33);
L17K(4-(nonanoylamino)butanoyl)-GLP-2(1-33);
L17K(4-(decanoylamino)butanoyl)-GLP-2(1-33);
L17K(4-(undecanoylamino)butanoyl)-GLP-2(1-33);
L17K(4-(dodecanoylamino)butanoyl)-GLP-2(1-33);
L17K(4-(tridecanoylamino)butanoyl)-GLP-2(1-33);
L17K(4-(tetradecanoylamino)butanoyl)-GLP-2(1-33);
L17K(4-(pentadecanoylamino)butanoyl)-GLP-2(1-33);
L17K(4-(hexadecanoylamino)butanoyl)-GLP-2(1-33);
L17K(4-(heptadecanoylamir1o)butanoyl)-GLP-2(1-33);
L17K(4-(octadecanoylamino)butanoyl)-GLP-2(1-33);
L17K(4-(nonadecanoylamino)butanoyl)-GLP-2(1-33);
L17K(4-(eicosanoylamino)butanoyl)-GLP-2(1-33);
A18K(3-(hexadecanoylamino)propionyl)-GLP-2(1-33);
D21K(3-(hexadecanoylamino)propionyl)-GLP-2(1-33);
N24K(3-(hexadecanoylamino)propionyl)-GLP-2(1-33);
Q28K(3-(hexadecanoylamino)propionyl)-GLP-2(1-33);
S5K(3-(hexadecanoylamino)propionyl)/K30R-GLP-2(1-33);
S7K(3-(hexadecanoylamino)propionyl)/K30R-GLP-2(1-33);
D8K(3-(hexadecanoylamino)propionyl)/K30R-GLP-2(1-33);
E9K(3-(hexadecanoylamino)propionyl)/K30R-GLP-2(1-33);
M10K(3-(hexadecanoylamino)propionyl)/K30R-GLP-2(1-33);
N11K(3-(hexadecanoylamino)propionyl)/K30R-GLP-2(1-33);
T12K(3-(hexadecanoylamino)propionyl)/K30R-GLP-2(1-33);
I13K(3-(hexadecanoylamino)propionyl)/K30R-GLP-2(1-33);
L14K(3-(hexadecanoylamino)propionyl)/K30R-GLP-2(1-33);
D15K(3-(hexadecanoylamino)propionyl)/K30R-GLP-2(1-33);
N16K(3-(hexadecanoylamino)propionyl)/K30R-GLP-2(1-33);
L17K(3-(octanoylamino)propionyl)/K30R-GLP-2(1-33);
L17K(3-(nonanoylamino)propionyl)/K30R-GLP-2(1-33);
L17K(3-(decanoylamino)propionyl)/K30R-GLP-2(1-33);
L17K(3-(undecanoylamino)propionyl)/K30R-G LP-2(1-33);
L17K(3-(dodecanoylamino)propionyl)/K30R-G LP-2(1-33);
L17K(3-(tridecanoylamino)propionyl)/K30R-G LP-2(1-33);
L17K(3-(tetradecanoylamino)propionyl)/K30R-GLP-2(1-33);
L17K(3-(pentadecanoylamino)propionyl)/K30R-GLP-2(1-33);
L17K(3-(hexadecanoylamino)propionyl)/K30R-GLP-2(1-33);
L17K(3-(heptadecanoylamino)propionyl)/K30R-GLP-2(1-33);
L17K(3-(octadecanoylamino)propionyl)/K30R-GLP-2(1-33);
L17K(3-(nonadecanoylamino)propionyl)/K30R-GLP-2(1-33);
L17K(3-(eicosanoylamino)propionyl)/K30R-G LP-2(1-33);
L17K((S)-carboxy-4-(octanoylamino)butanoyl)/K30R-G LP-2(1-33);
L17K((S)-4-carboxy-4-(nonanoylamino)butanoyl)/K30R-GLP-2(1-33);
L17K((S)-carboxy4-(decanoylamino)butanoyl)/K30R-GLP-2(1-33);
L17K((S)-4-carboxy-4-(undecanoylamino)butanoyl)/K30R-GLP-2(1-33);
L17K((S)-carboxy-4-(dodecanoylamino)butanoyl)/K30R-GLP-2(1-33);
L17K((S)-4-carboxy-4-(tridecanoylamino)butanoyl)/K30R-GLP-2(1-33);
L17K((S)-4-carboxy-4-(tetradecanoylamino)butanoyl)/K30R-GLP-2(1-33);
L17K((S)-4-carboxy-4-(pentadecanoylamino)butanoyl)/K30R-GLP-2(1-33):
L17K((S)-4-carboxy-4-(hexadecanoylamino)butanoyl)/K30R-GLP-2(1-33);
L17K((S)-4-carboxy-4-(heptadecanoylamino)butanoyl)/K30R-GLP-2(1-33);
L17K((S)-4-carboxy-4-(octadecanoylamino)butanoyl)/K30R-GLP-2(1-33);
L17K((S)-4-carboxy-4-(nonadecanoylamino)butanoyl)/K30R-GLP-2(1-33);
L17K((S)-4-carboxy-4-(eicosanoylamino)butanoyl)/K30R-GLP-2(1-33);
L17K(4-(octanoylamino)butanoyl)/K30R-GLP-2(1-33);
L17K(4-(nonanoylamino)butanoyl)/K30R-GLP-2(1-33);
L17K(4-(decanoylamino)butanoyl)/K30R-GLP-2(1-33);
L17K(4-(undecanoylamino)butanoyl)/K30R-GLP-2(1-33);
L17K(4-(dodecanoylamino)butanoyl)/K30R-GLP-2(1-33);
L17K(4-(tridecanoylamino)butanoyl)/K30R-GLP-2(1-33);
L17K(4-(tetradecanoylamino)butanoyl)/K30R-GLP-2(1-33);
L17K(4-(pentadecanoylamino)butanoyl)/K30R-GLP-2(1-33);
L17K(4-(hexadecanoylamino)butanoyl)/K30R-GLP-2(1-33);
L17K(4-(heptadecanoylamino)butanoyl)/K30R-GLP-2(1-33);
L17K(4-(octadecanoylamino)butanoyl)/K30R-GLP-2(1-33);
L17K(4-(nonadecanoylamino)butanoyl)/K30R-G LP-2(1-33);
L17K(4-(eicosanoylamino)butanoyl)/K30R-GLP-2(1-33);
A18K(3-(hexadecanoylamino)propionyl)/K30R-GLP-2(1-33);
D21K (3-(hexadecanoylamino)propionyl)/K30R-GLP-2(1-33);
N24K(3-(hexadecanoylamino)propionyl)/K30R-GLP-2(1-33);
Q28K(3-(hexadecanoylamino)propionyl)/K30R-GLP-2(1-33);
D3E/S5K(3-(hexadecanoylamino)propionyl)/K30R/D33E-GLP-2(1-33);
D3E/S7K(3-(hexadecanoylamino)propionyl)/K30R/D33E-GLP-2(1-33);
D3E/D8K(3-(hexadecanoylamino)propionyl)/K30R/D33E-GLP-2(1-33);
D3E/E9K(3-(hexadecanoylamino)propionyl)/K30R/D33E-GLP-2(1-33);
D3E/M10K(3-(hexadecanoylamino)propionyl)/K30R/D33E-GLP-2(1-33);
D3E/N11K (3-(hexadecanoylamino)propionyl)/K30/D33E-GLP-2(1-33);
D3E/T12K(3-(hexadecanoylamino)propionyl)/K30/D33E-GLP-2(1-33);
D3E/I13K(3-(hexadecanoylamino)propionyl)/K30R/D33E-GLP-2(1-33);
D3E/L14K(3-(hexadecanoylamino)propionyl)/K30R/D33E-GLP-2(1-33);
D3E/D15K(3-(hexadecanoylamino)propionyl)/K30R/D33E-GLP-2(1-33);
D3E/N16K(3-(hexadecanoylamino)propionyl)/K30R/D33E-GLP-2(1-33);
D3E/L17K(3-(octanoylamino)propionyl)/K30R/D33E-GLP-2(1-33);
D3E/L17K(3-(nonanoylamino)propionyl)/K30R/D33E-GLP-2(1-33);
D3E/L17K(3-(decanoylamino)propionyl)/K30/D33E-GLP-2(1-33);
D3E/L17K(3-(undecanoylamino)propionyl)/K30R/D33E-GLP-2(1-33);
D3E/L17K(3-(dodecanoylamino)propionyl)/K30R/D33E-GLP-2(1-33);
D3E/L17K(3-(tridecanoylamino)pr0pionyl)/K30R/D33E-GLP-2(1-33);
D3E/L17K(3-(tetradecanoylamino)propionyl)/K30R/D33E-GLP-2(1-33);
D3E/L17K(3-(pentadecanoylamino)propionyl)/K30R/D33E-GLP-2(1-33);
D3E/LI 7K(3-(hexadecanoylamino)propionyl)/K30R/D33E-GLP-2(1-33);
D3E/L17K(3-(heptadecanoylamino)propionyl)/K30R/D33E-GLP-2(1-33);
D3E/L17K(3-(octadecanoylamino)propionyl)/K30R/D33E-GLP-2(1-33);
D3E/L17K(3-(nonadecanoylamino)propionyl)/K30R/D33E-GLP-2(1-33);
D3E/L17K(3-(eicosanoylamino)pr0pionyl)/K30R/D33E-GLP-2(1-33);
D3E/L17K((S)-4-carboxy-4-(octanoylamino)butanoyl)/K30R/D33E-GLP-2(1-33);
D3E/L17K((S)-4-carboxy-4-(nonanoylamino)butanoyl)/K30R/D33E-GLP-2(1-33);
D3E/L17K((S)-4-carboxy-4-(decanoylamino)butanoyl)/K30R/D33E-GLP-2(1-33);
D3E/L17K((S)-4-carboxy-4-(undecanoylamino)butanoyl)/K30R/D33E-GLP-2(1-33);
D3E/L17K((S)-4-carboxy-4-(dodecanoylamino)butanoyl)/K30R/D33E-GLP-2(1-33);
D3E/L17K((S)-4-carboxy-4-(tridecanoylamino)butanoyl)/K30R/D33E-GLP-2(1-33);
D3E/L17K((S)-4-carboxy-4-(tetradecanoylamino)butanoyl)/K30R/D33E-GLP-2(1-33);
D3E/L17K((S)-4-carboxy-4-(pentadecanoylamino)butanoyl)/K30R/D33E-GLP-2(1-33);
D3E/L17K((S)-4-carboxy-4-(hexadecanoylamino)butanoyl)/K30R/D33E-GLP-2(1-33);
D3E/L17K((S)-4-carboxy-4-(heptadecanoylamino)butanoyl)/K30E/D33E-GLP-2(1-33);
D3E/L17K((S)-4-carboxy-4-(octadecanoylamino)butanoyl)/K30E/D33E-GLP-2(1-33);
D3E/L17K((S)-4-carboxy-4-(nonadecanoylamino)butanoyl)/K30R/D33E-GLP-2(1-33);
D3E/L17K((S)-4-carboxy-4-(eicosanoylamino)butanoyl)/K30R/D33E-GLP-2(1-33);
D3E/L17K(4-(octanoylamino)butanoyl)/K30R/D33E-GLP-2(1-33);
D3E/L17K(4-(nonanoylamino)butanoyl)/K30R/D33E-GLP-2(1-33);
D3E/L17K(4-(decanoylamino)butanoyl)/K30R/D33E-GLP-2(1-33);
D3E/L17K(4-(undecanoylamino)butanoyl)/K30R/D33E-GLP-2(1-33);
D3E/L17K(4-(dodecanoylamino)butanoyl)/K30R/D33E-GLP-2(1-33);
D3E/L17K(4-(tridecanoylamino)butanoyl)/K30R/D33E-G LP-2(1-33);
D3E/L17K(4-(tetradecanoylamino)butanoyl)/K30R/D33E-GLP-2(1-33);
D3E/L17K(4-(pentadecanoylamino)butanoyl)/K30R/D33E-GLP-2(1-33);
D3E/L17K(4-(hexadecanoylamino)butanoyl)/K30R/D33E-GLP-2(1-33);
D3E/L17K(4-(heptadecanoylamino)butanoyl)/K30R/D33E-GLP-2(1-33);
D3E/L17K(4-(octadecanoylamino)butanoyl)/K30R/D33E-GLP-2(1-33);
D3E/L17K(4-(nonadecanoylamino)butanoyl)/K30R/D33E-GLP-2(1-33);
D3E/L17K(4-(eicosanoylamino)butanoyl)/K30R/D33E-GLP-2(1-33);
D3E/A18K(3-(hexadecanoylamino)propionyl)/K30R/D33E-GLP-2(1-33);
D3E/D21K(3-(hexadecanoylamino)propionyl)/K30R/D33E-GLP-2(1-33);
D3E/N24K(3-(hexadecanoylamino)propionyl)/K30R/D33E-GLP-2(1-33); and
D3E/Q28K(3-(hexadecanoylamino)propionyl)/K30R/D33E-GLP-2(1-33).

Methods for the preparation of GLP-2, analogs thereof as well as GLP-2 derivatives can be found in e.g. WO 99/43361 and WO 00/55119.

In a further embodiment of the invention the glucagon-like peptide is an insulinotropic analog of exendin-4(1-39), e.g. Ser²Asp³-exendin-4(1-39) wherein the amino acid residues in position 2 and 3 have been replaced with serine and aspartic acid, respectively (this particular analog also being known-in the art as exendin-3).

In a further embodiment of the invention the glucagon-like peptide is an exendin-4 derivative wherein the substituent introduced is selected from amides, carbohydrates, alkyl groups, esters and lipophilic substituents. An example of insulinotropic derivatives of exendin-4(1-39) and analogs thereof is Tyr³¹-exendin4(1-31)-amide.

In one embodiment of the invention the glucagon-like peptide is a stable exendin-4 compound. In one embodiment of the invention the glucagon-like peptide is a DPP-IV protected exendin-4 compound. In one embodiment of the invention the glucagon-like peptide is an immunomodulated exendin-4 compound.

Methods for the preparation of exendin-4. analogs thereof as well as exendin-4 derivatives can be found in e.g. WO 99/43708, WO 00/41546 and WO 00/55119.

Pharmaceutical compositions containing a glucagon-like peptide purified according to the present invention typically contain various pharmaceutical excipients, such as preservatives, isotonic agents and surfactants. The preparation of pharmaceutical compositions is well known to the skilled person. For convenience reference is made to Remington: The Science and Practice of Phamacy, 1grn edition, 1995.

Pharmaceutical compositions containing a glucagon-like peptide purified according to the present invention may be administered parenterally to patients in need of such treatment. Parenteral administration may be performed by subcutaneous injection, intramuscular injection, or intraveneous injection by means of a syringe, optionally a pen-like syringe. Alternatively, administration can be performed by infusion, e.g. by use of an infusion pump.

The following is a list of embodiments according to the invention:

1. A process for purifying a peptide prepared by solid phase peptide synthesis which comprises the step of bringing a crude extract of the peptide prepared by solid phase peptide synthesis in contact with a solid support.
2. A process according to embodiment 1, wherein solid phase peptide synthesis comprises the use of Fmoc as an amino-terminal protecting group.
3. A process according to embodiment 1 or 2 wherein said process comprises a process for removing dibenzofulvene from the crude peptide extract.
4. A process according to any of embodiments 1 to 3, wherein the solid support comprises a packaging material comprising a thermoplastic polymer.
5. A process according to embodiment 4, wherein the solid support is selected from the group consisting of a container, pellets, particles and a filter-support.
6. A process according to embodiment 4 or 5, wherein the thermoplastic polymer is polyethylene or polypropylene.
7. A process according to embodiment 6, wherein the thermoplastic polymer is polyethylene.
8. A process according to any of embodiments 4 to 7, which comprises the following steps:
(a) addition of the crude peptide extract obtained from solid-phase synthesis to the packaging material;
(b) incubation of the extract in the packaging material;
(c) removal of the extract from the packaging material; and
(d) subjecting the extract to standard peptide separation.
9. A process according to embodiment 8, wherein the polyethylene is high density polyethylene (HDPE).
10. A process according to embodiment 8 or 9, wherein the incubation step (b) comprises incubation at an ambient temperature for a duration of between 2 minutes and 10 hours.
11. A process according to any of embodiments 8 to 10, wherein the incubation step (b) comprises incubation at room temperature.
12. A process according to any of embodiments 8 to 11, wherein step (b) additionally comprises agitation of the extract.
13. A process according to any of embodiments 8 to 12, wherein the standard peptide separation in step (d) comprises chromatographic separation which includes batch absorption, a packed column or a filter.
14. A process according to any of embodiments 8 to 13, wherein the standard peptide separation in step (d) comprises chromatographic separation wherein a packed column or a filter is used, and wherein the residence time is at least 0.1 minutes.
15. A process according to any of embodiments 8 to 14, wherein the standard peptide separation in step (d) is ion-exchange chromatography.
16. A process according to any of embodiments 1 to 3, wherein the solid support comprises an ion-exchange chromatographic column.
17. A process according to embodiment 16, wherein the solid support comprises an anionic resin.
18. A process according to embodiment 17, wherein the anionic resin is a quaternary ammonium resin.
19. A process according to any of embodiments 16 to 18 which comprises the following steps:
(a) under standard chromatographic conditions loading the ion-exchange chromatographic column with the crude peptide extract obtained from solid-phase synthesis or the peptide obtained from steps (a) to (c) in the process of embodiment 8;
(b) performing a first elution step with a an alcohol; and
(c) performing a second elution step with one or more buffers.
20. A process according to embodiment 19 wherein the buffers used in step (c) include Tris(tris(hydroxymethyl)methylamine), TAPS(3-{[tris(hydroxymethyl)methyl]amino}propanesulfonic acid), Bicine(N,N-bis(2-hydroxyethyl)glycine), Tricine(N-tris(hydroxymethyl)methylglycine), HEPES(4-2-hydroxyethyl-1-piperazineethanesulfonic acid), TES(2-{[tris(hydroxymethyl)methyl]amino}ethanesulfonic acid), MOPS(3-(N-morpholino)propanesulfonic acid), PIPES(piperazine-N,N′-bis(2-ethanesulfonic acid)), Cacodylate(dimethylarsinic acid), MES(2-(N-morpholino)ethanesulfonic acid) or acetate.
21. A process according to embodiment 20 wherein the buffer used in step (c) is Tris buffer.
22. A process according to any of embodiments 19 to 21 wherein the alcohol used in step (b) is a C₁₋₅ alcohol.
23. A process according to any of embodiments 19 to 22 wherein the alcohol used in step (b) is an unbranched or branched alcohol selected from the group consisting of: methanol, ethanol, 1-propanol, 2-propanol, 2-methyl-1-propanol, 2-methyl-2-propanol, 1-butanol, 2-butanol, 2-methyl-1-butanol, 3-methyl-1-butanol, 2-methyl-2-butanol, 3-methyl-2-butanol, 1-pentanol, 2-pentanol and 3-pentanol.
24. A process according to any of embodiments 19 to 23 wherein the alcohol used in step (b) is ethanol or propanol.
25. A process according to any of embodiments 19 or 24 wherein the alcohol used in step (b) is selected from the group consisting of 70% ethanol, 80% ethanol, 90% ethanol or 100% ethanol.
26. A process according to any of embodiments 19 or 25 wherein the alcohol used in step (b) is 100% ethanol.
27. A process according to any preceding embodiments, wherein the polypeptide is a glucagon-like peptide.
28. A process according to embodiment 27, wherein the polypeptide is glucagon, a glucagon analogue, a derivative of glucagon or a derivative of a glucagon analogue.
29. A process according to embodiment 27, wherein the glucagon-like peptide is GLP-1, a GLP-1 analogue, a derivative of GLP-1 or a derivative of a GLP-1 analogue.
30. A solid phase peptide synthesis kit which comprises reagents for solid phase peptide synthesis, a solid support as defined in any of embodiments 1 to 29 and instructions to use said kit in accordance with the process as defined in any of embodiments 1 to 29.
31. A peptide obtained by a process described in any of embodiments 1 to 29.

All references, including publications, patent applications and patents, cited herein are hereby incorporated by reference to the same extent as if each reference was individually and specifically indicated to be incorporated by reference and was set forth in its entirety herein.

All headings and sub-headings are used herein for convenience only and should not be construed as limiting the invention in any way.

Any combination of the above-described elements in all possible variations thereof is encompassed by the invention unless otherwise indicated herein or otherwise clearly contradicted by context.

The terms “a” and “an” and “the” and similar referents as used in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate). All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise indicated. No language in the specification should be construed as indicating any element is essential to the practice of the invention unless as much is explicitly stated.

The citation and incorporation of patent documents herein is done for convenience only and does not reflect any view of the validity, patentability and/or enforceability of such patent documents,

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a formulation described herein as comprising a particular element should be understood as also describing a formulation consisting of that element, unless otherwise stated or clearly contradicted by context).

This invention includes all modifications and equivalents of the subject matter recited in the aspects or claims presented herein to the maximum extent permitted by applicable law.

The present invention is further illustrated by the following examples which, however, are not to be construed as limiting the scope of protection. The features disclosed in the foregoing description and in the following examples may, both separately and in any combination thereof, be material for realizing the invention in diverse forms thereof.

### EXAMPLES

### Equipment:

ÄKTA Explorer100 at room temperature.

### Buffers:

Buffer A: 50% (w/w) EtOH, 0.02 mol/kg Tris, pH 8.0
Buffer B: 50% (w/w) EtOH, 0.02 mol/kg Tris, 0.0625 mol/kg NaCl, pH 8.0

### Regeneration 1:1 M NaOH

### Regeneration 2: 2 M NaCl, 50 mM CH₃COOH, pH 3, 0

### Ethanol: 100% Ethanol

### Resin:

### Source 30Q

### Starting Material:

N-epsilon26-[2-(2-[2-(2-[2-(2-[4-(17-carboxyheptadecanoylamino)-4(S)-carboxybutyrylamino]ethoxy)ethoxy]acetylamino)ethoxy]ethoxy)acetyl][Aib8,Arg34]GLP-1-[7-37] produced by solid phase synthesis and acylation.

### Example 1

### Control Purification Procedure

### Preparation of Starting Material

Starting material were diluted 1+9 with H₂O (WFI) followed by filtration through 0.6/0.2 μm filter.

### Storage of Starting Material

Starting material stored in glass container prior to loading.

### Method:

Column: HR10/10 (3.5.1.0 cm) 2.8 mL

Flow: 15 CV/h (0.7 mL/min)

| Segment | Volume and buffer |
|---|---|
| Equilibration | 10 CV Buffer A |
| Load | 2 CV (5.6 ml) ~ 2 g N-ε²⁶-[2-(2-[2-(2-[2-(2-[4-(17- |
| | Carboxyheptadecanoylamino)-4(S)- |
| | carboxybutyrylamino]ethoxy)ethoxy]acetylamino)ethoxy] |
| | ethoxy)acetyl][Aib⁸, Arg³⁴]GLP-1-(7-37)peptide /L resin |
| Wash | 7 CV Buffer A |
| Elution 1 | Linear gradient from 100% Buffer A and 0% Buffer B to 50% |
| | Buffer A and 50% Buffer B over 20 CV |
| Elution 2 | 5 CV Buffer B |
| Regeneration | 5 CV Regeneration 1 |
| 1 | |
| Regeneration | 5 CV Regeneration 2 |
| 2 | |

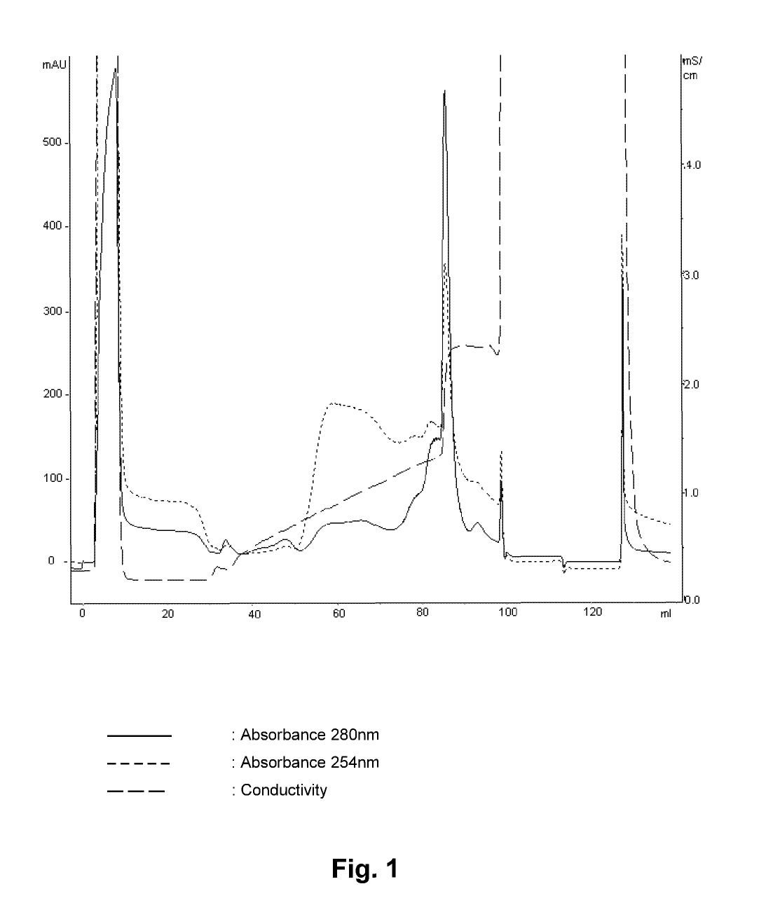

The results of the standard separation are shown in the chromatogram of FIG. 1, wherein the chromatogram demonstrates absorbance at 280 nm, absorbance at 254 nm, theoretical gradient and conductivity. The results from FIG. 1 show that the DBF peak elutes prior to the product but is “trailing” under the product and is therefore hard to remove.

### Example 2

### Purification by Anion Exchange after PE-Treatment (Including Elution of DBF from PE-Container)

### Preparation of Starting Material

The starting material was prepared as described in Example 1.

### Storage of Starting Material

Starting material stored in HDPE (high density polyethylene) container (“Mellerud container” obtained from Emballator) prior to loading.

### Method:

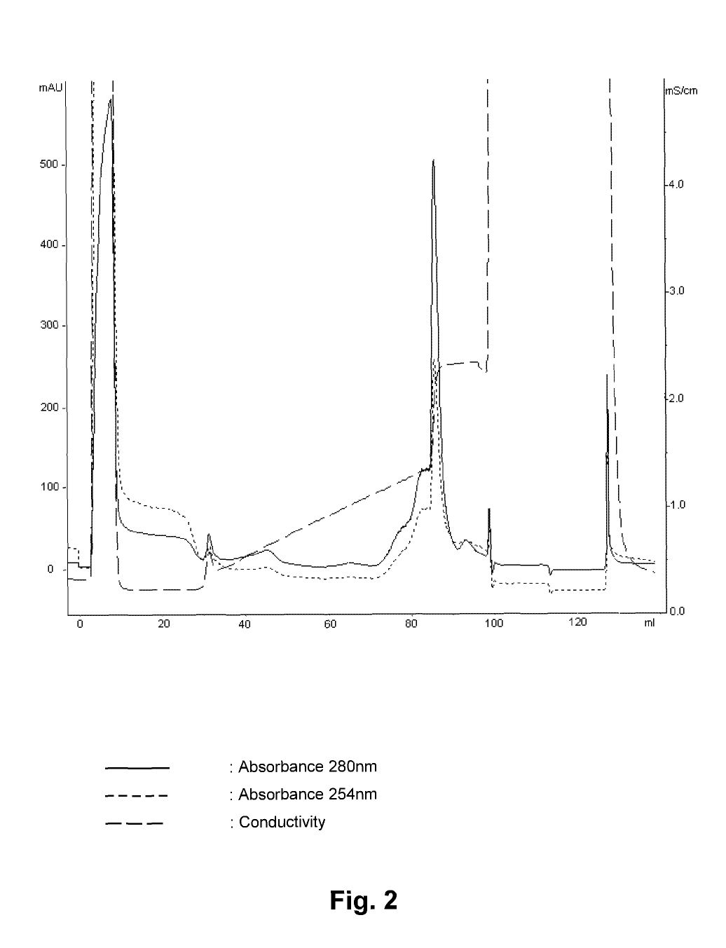

The method was performed as described in Example 1 and the results are shown in FIG. 2, wherein the chromatogram demonstrates absorbance at 280 nm, absorbance at 254 nm, theoretical gradient and conductivity. In view of the fact that the experiment is identical to Example 1 with the exception of the sample storage conditions, the DBF peak appears to be absent due to the storage of the starting material in the HDPE container prior to loading.

The Mellerud container was washed with 100% ethanol after the starting material was removed because it was assumed that DBF bound hydrophobic to PE and that it would therefore be possible to remove DBF with a hydrophobic liquid. An absorbance measurement of the washing solution clearly showed that the DBF had bound to the container and could be desorped or “eluted” with 100% ethanol from the container.

### Example 3

### Anion Exchange Purification with Ethanol Wash after Loading

### Preparation of Starting Material

Dilution 10 times in H₂O (from 10 g starting material to 1 g starting material) followed by pH adjustment from pH 6 to pH 8.0 with NaOH. The starting material was then filtered using 0.22 μm filter.

### Storage of Starting Material

Starting material stored in glass container prior to loading.

### Method:

Column: 15.1.0 cm) 11.8 mL

Flow 15 CV/h (0.7 mL/min)

| Segment | Volume and buffer |
|---|---|
| Equilibration | 3 CV Buffer A |
| Load | 3 CV (5.6 ml)~2.5 g N-ε²⁶-[2-(2-[2-(2-[2-(2-[4-(17- |
| | Carboxyheptadecanoylamino)-4(S)- |
| | carboxybutyrylamino]ethoxy)ethoxy]acetylamino)ethoxy] |
| | ethoxy)acetyl][Aib⁸,Arg³⁴]GLP-1-(7-37)peptide/L resin |
| Wash 1 | 2 CV Buffer A |
| Wash 2 | 3 CV Ethanol |
| Wash 3 | 2 CV Buffer A |
| Elution 1 | Linear gradient from 100% Buffer A and 0% Buffer B to |
| | 60% Buffer A and 40% Buffer B over 16 CV |
| Elution 2 | Linear gradient from 60% Buffer A and 40% Buffer B to |
| | 0% Buffer A and 100% Buffer B over 2 CV |
| Elution 3 | 10 CV Buffer B |
| Regeneration 1 | 3 CV Regeneration 1 |
| Regeneration 2 | 3 CV Regeneration 2 |

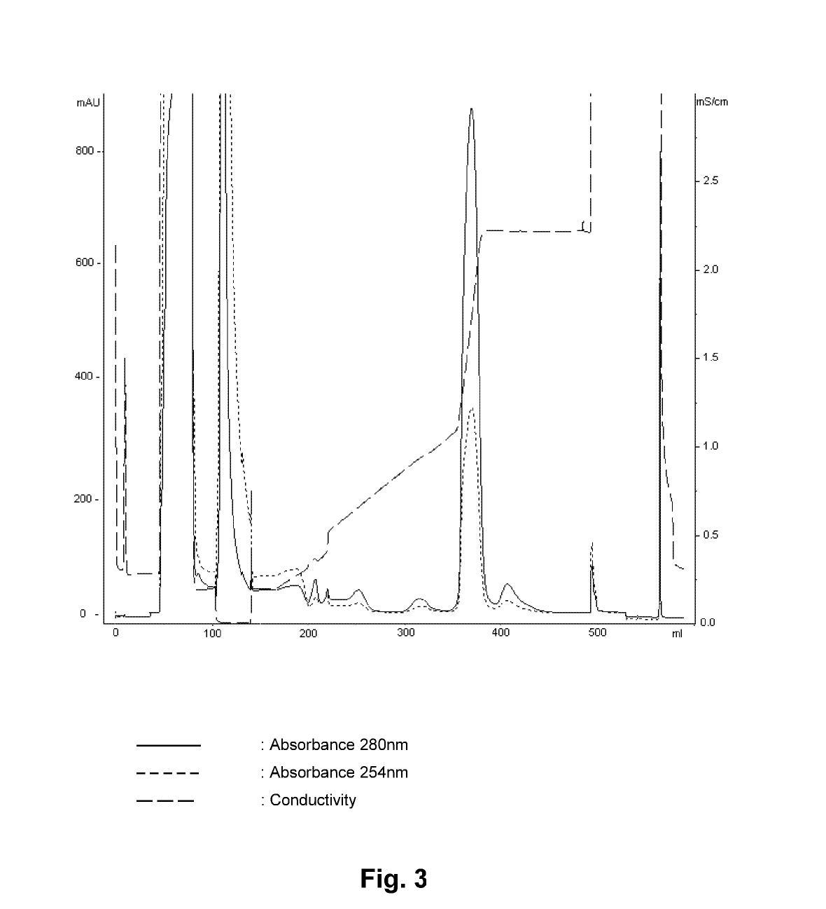

The results are shown in FIG. 3, wherein the chromatogram demonstrates absorbance at 280 nm, absorbance at 254 nm, theoretical gradient and conductivity. The results demonstrate that the DBF peak elutes during the ethanol wash.

1. A process for purifying a peptide prepared by solid phase peptide synthesis, the process comprising bringing a crude extract of the peptide prepared by solid phase peptide synthesis in contact with a solid support, wherein solid phase peptide synthesis comprises the use of Fmoc as an amino-terminal protecting group and wherein said process removes dibenzofulvene from the crude peptide extract.

2-3. (canceled)

4. A process according to claim 1, wherein the solid support comprises a packaging material comprising a thermoplastic polymer.

5. A process according to claim 4, wherein the solid support is selected from the group consisting of a container, pellets, particles and a filter-support.

6. A process according to claim 5, wherein the thermoplastic polymer is polyethylene or polypropylene.

7. A process according to claim 4, which comprises the following steps:
(a) addition of the crude peptide extract obtained from solid-phase synthesis to the packaging material;
(b) incubation of the extract in the packaging material;
(c) removal of the extract from the packaging material; and
(d) subjecting the extract to standard peptide separation.

8. A process according to claim 7 wherein the standard peptide separation in step (d) is ion-exchange chromatography.

9. A process according to claim 1, wherein the solid support comprises an ion-exchange chromatographic column.

10. A process according to claim 9, wherein the solid support comprises an anion-exchange chromatographic column.

11. A process according to claim 9 which comprises the following steps:
(a) under standard chromatographic conditions loading the ion-exchange chromatographic column with the crude peptide extract obtained from solid-phase synthesis or the peptide obtained from steps (a) to (c) in the process of claim 7;
(b) performing a first elution step with an alcohol; and
(c) performing a second elution step with one or more buffers.

12. A process according to claim 11 wherein the buffers used in step (c) include Tris(tris(hydroxymethyl)methylamine), TAPS(3-{[tris(hydroxymethyl)methyl]amino}propanesulfonic acid), Bicine(N,N-bis(2-hydroxyethyl)glycine), Tricine(N-tris(hydroxymethyl)methylglycine), HEPES(4-2-hydroxyethyl-1-piperazineethanesulfonic acid), TES(2-{[tris(hydroxymethyl)methyl]amino}ethanesulfonic acid), MOPS(3-(N-morpholino)propanesulfonic acid), PIPES(piperazine-N,N′-bis(2-ethanesulfonic acid)), Cacodylate(dimethylarsinic acid), MES(2-(N-morpholino)ethanesulfonic acid) or acetate.

13. A process according to claim 11 wherein the alcohol used in step (b) is a C₁₋₅ alcohol.

14. A process according to, claim 1 wherein the polypeptide is a glucagon-like peptide.

15. A peptide obtained by a process according to claim 1.


SEQUENCE LISTING (6 sequences)

SEQ ID NO: 1 - protein, 29 aa, Homo sapiens
  MOD_RES (29)..(29): AMIDATION
HSQGTFTSDYSKYLDSRRAQDFVQWLMNT

SEQ ID NO: 2 - protein, 31 aa, Homo sapiens
HAEGTFTSDVSSYLEGQAAKEFIAWLVKGRG

SEQ ID NO: 3 - protein, 33 aa, Homo sapiens
HADGSFSDEMNTILDNLAARDFINWLIQTKITD

SEQ ID NO: 4 - protein, 39 aa, Heloderma suspectum
  MOD_RES (39)..(39): AMIDATION
HGEGTFTSDLSKQMEEEAVRLFIEWLKNGGPSSGAPPPS

SEQ ID NO: 5 - protein, 39 aa, Heloderma suspectum
  MOD_RES (39)..(39): AMIDATION
HSDGTFTSDLSKQMEEEAVRLFIEWLKNGGPSSGAPPPS

SEQ ID NO: 6 - protein, 37 aa, Homo sapiens
HSQGTFTSDYSKYLDSRRAQDFVQWLMDTKRNKNNIA